(19) United States
(12) Patent Application Publication
(10) Pub. No.: US 2026/0137546 A1
(43) Pub. Date: May 21, 2026

(54) PAD FOR COLLECTING AND DIRECTING URINE

(71) Applicant: Ana Mendez, Sacramento, CA (US)

(72) Inventor: Ana Mendez, Sacramento, CA (US)

(21) Appl. No.: 19/391,288

(22) Filed: Nov. 17, 2025

Related U.S. Application Data
(60) Provisional application No. 63/721,638, filed on Nov. 18, 2024.

(51) Int. Cl. A61F 5/44

(52) U.S. Cl. CPC A61F 5/4405; A61F 5/4401

(57) ABSTRACT

A pad for collecting and directing urine of a wearer is disclosed. The pad includes a panel body having an exit port to allow an exit of urine. The pad also includes a valve attached to panel body and arranged to receive urine from the panel body. The valve enables an engagement of an external tube to direct the urine received from the panel body via the exit port to an external bag. The pad also includes a ring structure extending along a periphery of the panel body and adapted to contact a skin of the wear to provide a sealing between the pad and the skin to prevent any leakage of the urine from the pad.

### CROSS REFERENCE TO RELATED APPLICATIONS

This application claims priority to and the benefit of U.S. Provisional Patent Application No. 63/721,638, filed Nov. 18, 2024, which is incorporated herein by reference.

### TECHNICAL FIELD

The present disclosure relates, generally, to pad. More particularly, the present disclosure pertains to a pad adapted to be worn by a user to direct urine to an external bag.

### BACKGROUND

With the advancement of modern medical science, improved nutrition, and enhanced living conditions, human life expectancy has significantly increased. However, aging is often accompanied by various physiological changes that affect the normal functioning of the urinary and excretory systems. One of the most prevalent issues among older adults is the loss of control over urination and defecation, which may result from weakened pelvic floor muscles, neurological disorders, or chronic medical conditions.

Conventional solutions for managing these problems—such as adult diapers, bedpans, catheters, and absorbent pads—often fail to adequately address the practical and comfort needs of users. These devices can lead to discomfort, skin irritation, leakage, unpleasant odors, and an increased risk of infection.

In addition, caregivers and healthcare personnel frequently encounter difficulties in monitoring, cleaning, and maintaining these systems, resulting in increased workload and potential hygiene and infection control challenges.

### SUMMARY

In one aspect of the disclosure, a pad for collecting and directing urine of a wearer is disclosed. The pad includes a panel body having an exit port to allow an exit of urine. The pad also includes a valve attached to panel body and arranged to receive urine from the panel body. The valve enables an engagement of an external tube to direct the urine received from the panel body via the exit port to an external bag. The pad also includes a ring structure extending along a periphery of the panel body and adapted to contact a skin of the wear to provide a sealing between the pad and the skin to prevent any leakage of the urine from the pad.

In some additional, alternative, or selectively cumulative embodiments, the panel body defines a plurality of grooves defining a plurality of channels to direction the urine to the exit port.

In some additional, alternative, or selectively cumulative embodiments, the ring structure is integrally formed with the panel body and extends upwardly from the panel body defining a chamber to retain the urine.

In some additional, alternative, or selectively cumulative embodiments, the pad further includes an outer layer arranged engaged to the panel body. The outer layer is a perforated layer to allow a flow the urine received from the wearer to the panel body.

In some additional, alternative, or selectively cumulative embodiments, the outer layer is integrally formed with the panel body.

In some additional, alternative, or selectively cumulative embodiments, the outer layer is removably attached to the panel body.

In some additional, alternative, or selectively cumulative embodiments, the panel body includes a retention structure and the outer layer includes an engagement structure configured to removably engage with the retention structure to removably engage the outer layer with the panel body.

In some embodiments, an edge portion of the outer layer is inserted partially underneath the ring structure to form a mechanical seal and prevent leakage between the fabric and the panel body.

In some additional, alternative, or selectively cumulative embodiments, the engagement structure is a slot and the retention structure is a bead adapted to extend inside the slot.

In some additional, alternative, or selectively cumulative embodiments, the ring structure is attached to the outer layer and extends along an outer edge of the outer layer.

In some additional, alternative, or selectively cumulative embodiments, the valve is removably attached to the panel body.

### BRIEF DESCRIPTION OF THE DRAWINGS

FIG. 1 illustrates a pad arranged supported in a crotch portion of an underwear and the pad connected with an external bag via an external tube to collect urine, in accordance with an embodiment of the present disclosure;

FIG. 2 illustrates a portion of FIG. 1 depicting the external tube extending from the pad through the underwear, in accordance with an embodiment of the present disclosure;

FIG. 3A illustrates a top perspective view of an example pad, in accordance with an embodiment of the present disclosure;

FIG. 3B illustrates a sectional of the pad of FIG. 3A, in accordance with an embodiment of the present disclosure;

FIG. 4 illustrates a top perspective view of a panel body of the pad of FIG. 3A, in accordance with an embodiment of the present disclosure;

FIG. 5 illustrates a sectional view of the panel body of FIG. 4, in accordance with an embodiment of the present disclosure;

FIG. 6 illustrates a bottom perspective view of the panel body of FIG. 4, in accordance with an embodiment of the present disclosure;

FIG. 7 illustrates a top perspective view of a pad, in accordance with an embodiment of the present disclosure;

FIG. 8 illustrates an exploded view of the pad of FIG. 7, in accordance with an embodiment of the present disclosure;

FIG. 9 illustrates an exploded view of the pad of FIG. 8, in accordance with an embodiment of the present disclosure;

FIG. 10 illustrates a top perspective view of a pad depicting various layer of the pad, in accordance with an embodiment of the present disclosure; and

FIG. 11 illustrates a bottom perspective view of a pad depicting various layer of the pad, in accordance with an embodiment of the present disclosure.

### DETAILED DESCRIPTION

Example embodiments are described below with reference to the accompanying drawings. Unless otherwise expressly stated in the drawings, the sizes, positions, etc., of components, features, elements, etc., as well as any distances therebetween, are not necessarily to scale, and may be disproportionate and/or exaggerated for clarity.

The terminology used herein is for the purpose of describing example embodiments only and is not intended to be limiting. As used herein, the singular forms “a,” “an” and “the” are intended to include the plural forms as well, unless the context clearly indicates otherwise. It should be recognized that the terms “comprise,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise specified, a range of values, when recited, includes both the upper and lower limits of the range, as well as any sub-ranges therebetween. Unless indicated otherwise, terms such as “first,” “second,” etc., are only used to distinguish one element from another. For example, one element could be termed a “first element” and similarly, another element could be termed a “second element,” or vice versa. The section headings used herein are for organizational purposes only and are not to be construed as limiting the subject matter described.

Unless indicated otherwise, the terms “about,” “thereabout,” “substantially,” etc., mean that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art.

Spatially relative terms, such as “right,” “left,” “below,” “beneath,” “lower,” “above,” and “upper,” and the like, may be used herein for ease of description to describe one element's or feature's relationship to another element or feature, as illustrated in the drawings. It should be recognized that the spatially relative terms are intended to encompass different orientations in addition to the orientation depicted in the figures. For example, if an object in the figures is turned over, elements described as “below” or “beneath” other elements or features would then be oriented “above” the other elements or features. Thus, the term “below” can, for example, encompass both an orientation of above and below. An object may be otherwise oriented (e.g., rotated 90 degrees or at other orientations) and the spatially relative descriptors used herein may be interpreted accordingly.

Unless clearly indicated otherwise, all connections and all operative connections may be direct or indirect. Similarly, unless clearly indicated otherwise, all connections and all operative connections may be rigid or non-rigid.

Like numbers refer to like elements throughout. Thus, the same or similar numbers may be described with reference to other drawings even if they are neither mentioned nor described in the corresponding drawing. Also, even elements that are not denoted by reference numbers may be described with reference to other drawings.

Many different forms and embodiments are possible without deviating from the spirit and teachings of this disclosure and so this disclosure should not be construed as limited to the example embodiments set forth herein. Rather, these example embodiments are provided so that this disclosure will be thorough and complete, and will convey the scope of the disclosure to those skilled in the art.

Reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments.

Reference will now be made in detail to specific embodiments or features, examples of which are illustrated in the accompanying drawings. Generally, corresponding reference numbers will be used throughout the drawings to refer to the same or corresponding parts. Also, wherever possible, the same reference numbers will be used throughout the drawings to refer to the same or the like parts.

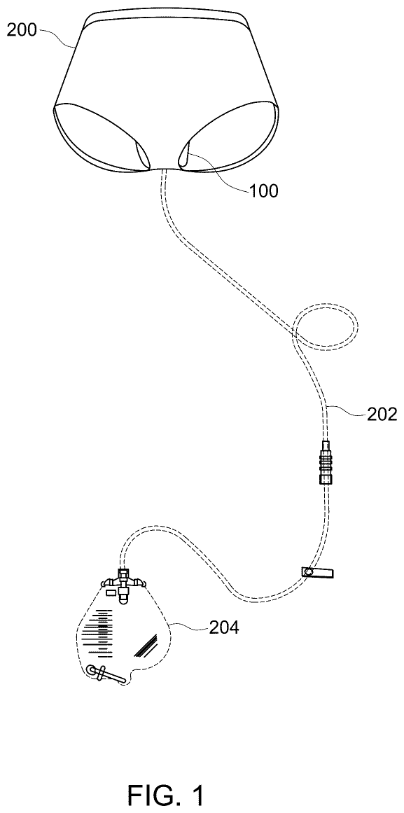

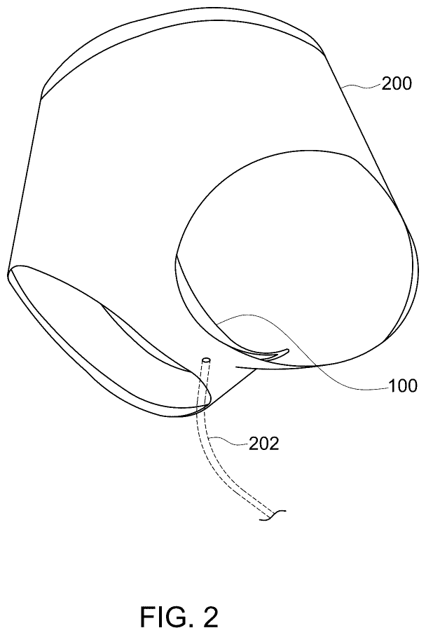

Referring to FIGS. 1 and 2, a pad 100 removably attached to an underwear 200 suitable to be worn by a user is shown according to example embodiments of the disclosure. In the embodiments, the pad 100 is arranged supported on crotch portion of the underwear 200 and is arranged to collect and direct urine discharged by the person. In the embodiments, an external tube 202 is shown to be attached to the pad 100 to direct the urine from the pad 100 to a bag 204 for collection of the urine.

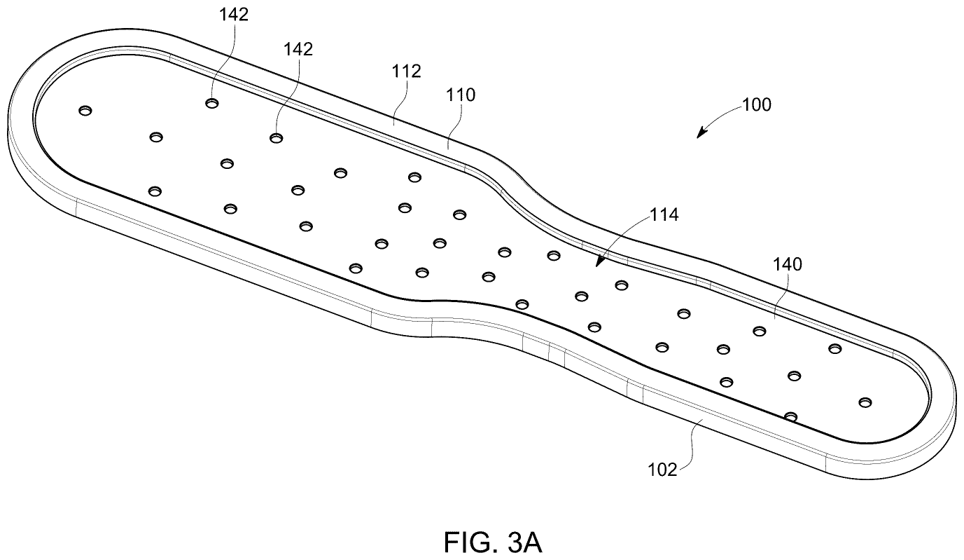

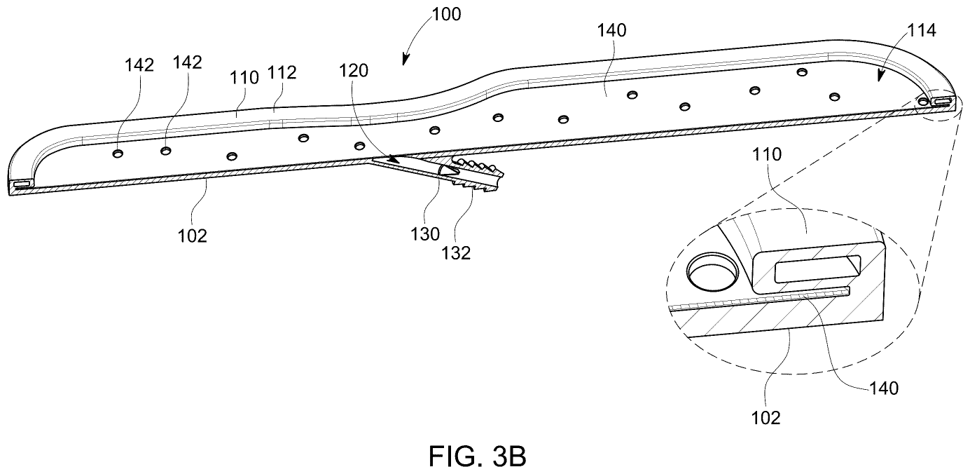

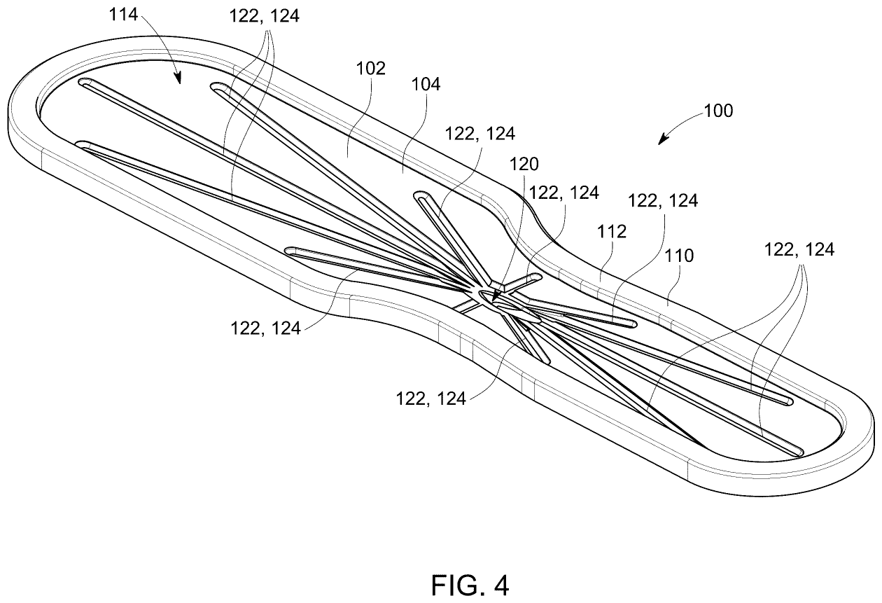

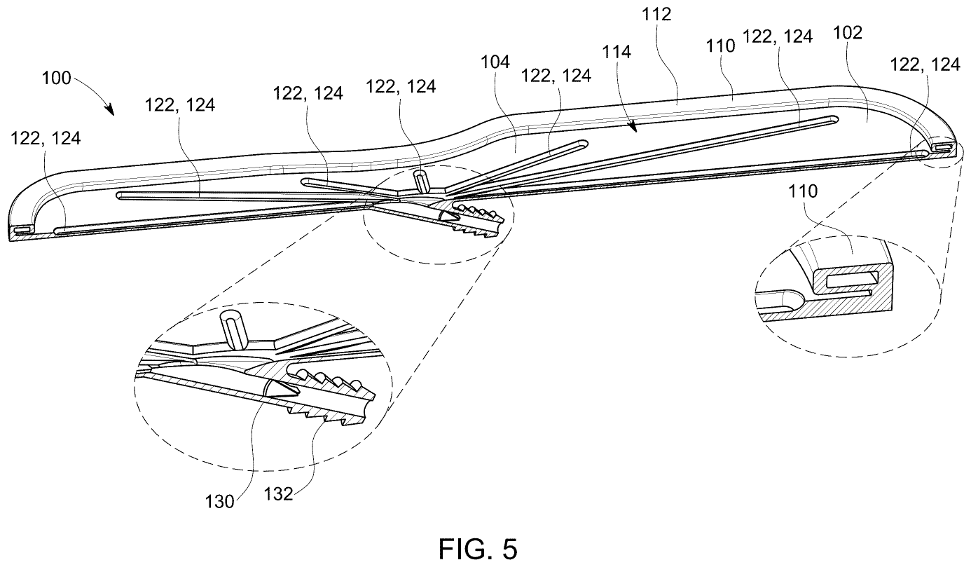

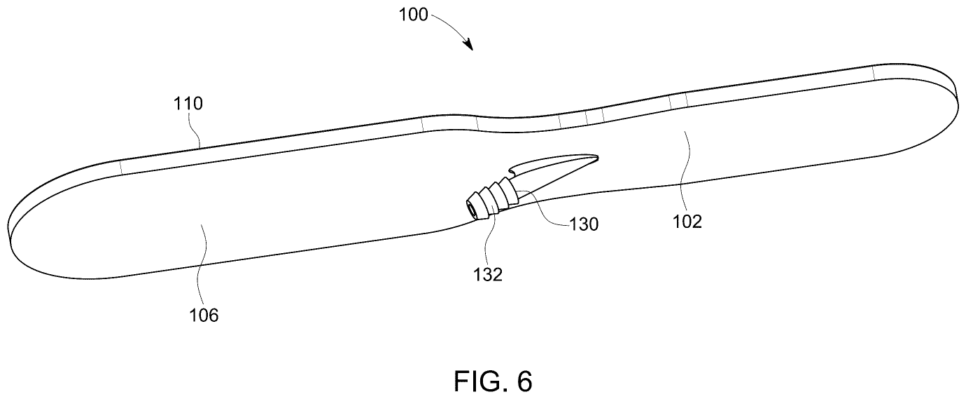

Referring to FIGS. 3A to 6, the example pad 100 is shown, according to some embodiments of the disclosure. As shown in FIGS. 3A to 6, the pad 100 includes a panel body 102 having a first surface 104, shown in FIGS. 4 and 5, and a second surface 106, shown in FIG. 6, arranged opposite to the first surface 104, and a ring structure 110 arranged connected to the panel body 102 and extending along an outer edge of the panel body 102, surrounding the panel body 102. In the embodiment, the ring structure 110 extends vertically upwardly of the first surface 104 such that an upper surface 112 of the ring structure 110 is arranged at a suitable height from the first surface 104 of the panel body 102. Accordingly, a urine collection chamber 114 is defined between the first surface 104 and the ring structure 110 in which urine is temporarily retained. In some embodiments, the panel body 102 may be anatomically contoured to follow the perineal curvature to improve comfort and sealing performance.

To facilitate removal of the urine or any other discharge from the chamber 114, the panel body 102 defines an exit port 120 extending from the first surface 104 to the second surface 104. In the illustrate embodiment, the exit port 120 is arranged substantially centrally to the panel body 102. Further, to direct the urine toward the exit port 120, the panel body 102 defines a plurality of elongated grooves 122 extending downwardly from the first surface 104 towards the second surface 106. Each of the plurality of grooves 122 defines an elongated channel 124 with one end of the channel 124 arranged connected to the exit port 120. Therefore, the urine present inside the chamber 114 flows to the exit port 120 via the plurality of elongated channels 124. As shown, the elongated channels 124 are open from top.

Further, to facilitate a connection of the external tube 202 with the pad 100 and to allow a flow of the urine from the pad 100 to the external collection bag 204, the pad 100 includes a valve 130, best shown in FIGS. 5 and 6, extending outwardly and downwardly from the second surface 106 of the panel body 102. The valve 130 is arranged at the location of the exit port 120 and is arranged such that the urine enters the valve 130 through the exit port 120. In the embodiment, an outer surface of the valve 130 includes a plurality of ribs 132 or serrations to enable a secure engagement of the external tube 202 with the valve 130, and prevents any undesired disengagement of the external tube 202 from the pad 100. In the embodiments, the valve 130 is integrally formed with the panel body 102, and extends at an acute angle from the second surface 106 of the panel body 102. In embodiments, the valve 130 may be removably attached to the panel body 102. In some embodiments, the valve 130 is a one-way valve and prevents a back flow the urine from the external tube 202.

The ring structure 110 acts as a sealing ring adapted to engage with a skin of the wearer, and prevents a leakage of urine or discharge collected inside the chamber. In embodiments, the ring structure 110 includes a shape to increase a surface area of the upper surface 112 of the ring structure 110 that is arranged engaged with the skin of the wearer. In the illustrated embodiment, shown in FIGS. 3B and 5, the ring structure 110 includes a substantially rectangular cross section, however, the ring structure 110 may include any other cross-section shape, such as, but not limited to, square, trapezoidal, or any other shape suitable to provide adequate engagement surface to provide a good sealing with the skin surface. In embodiments, to enhance the sealing action of ring structure 110, the ring structure 100 is made of a flexible or compressible material, such as, but not limited to, silicone, polyurethane, while provide structural integrity to the pad. In embodiments, the ring structure is integrally formed with the panel body.

In some embodiments, the panel body 102, the ring structure 110, and the valve 130, each may be formed a flexible, biocompatible, and skin-contact safe material, such as, but not limited to thermoplastic polyurethane (TPU), thermoplastic elastomers (TPE), silicone, or any other similar material known in the art.

In some additional, or optional embodiments, as shown in FIGS. 3A and 3B, the pad 100 includes a cover layer 140 i.e., outer layer arranged facing the first surface 102 of the panel body 102 and attached to the panel body 102 and/or the ring structure 110. To facilitate the flow of urine to the chamber 114 through the cover layer 140, the cover layer 140 is a perforated layer having a plurality of through holes 142. The cover layer 140 may be integrally formed with the panel body 102 and/or the ring structure 110. In embodiments, the cover layer 140 is made of fabric. In some embodiments, the cover layer 140 is made of liquid-permeable and quick-dry mesh/knit material such as, but not limited to, polyester micro-mesh, CoolMax®, ProCool TransWICK®, nylon knit, or any other similar material known in the art. In some embodiments, the cover layer 140 may be omitted. In some embodiments, the cover layer 140 may be a replaceable and removable layer. In such a case, in some embodiments, an edge portion of the cover layer 140 is inserted partially underneath the ring structure 110, as shown in FIG. 3B, to form a mechanical seal and prevent leakage between the cover layer 140 and the panel body 102.

In some embodiments, the pad 100 includes a total length of approximately 17 inches, a posterior width of approximately 4 inches, an anterior width of approximately 3 inches, a tunnel width of approximately 0.45 inches, and a tunnel height of approximately 0.1875 inches. It may be appreciated that the provided dimension are only exemplary dimensions of the pad and does not limit the scope of the disclosure Further, it may be envisioned that a size and hence dimensions of the pad may be adjusted based on different body types or age groups, including adolescent or pediatric users.

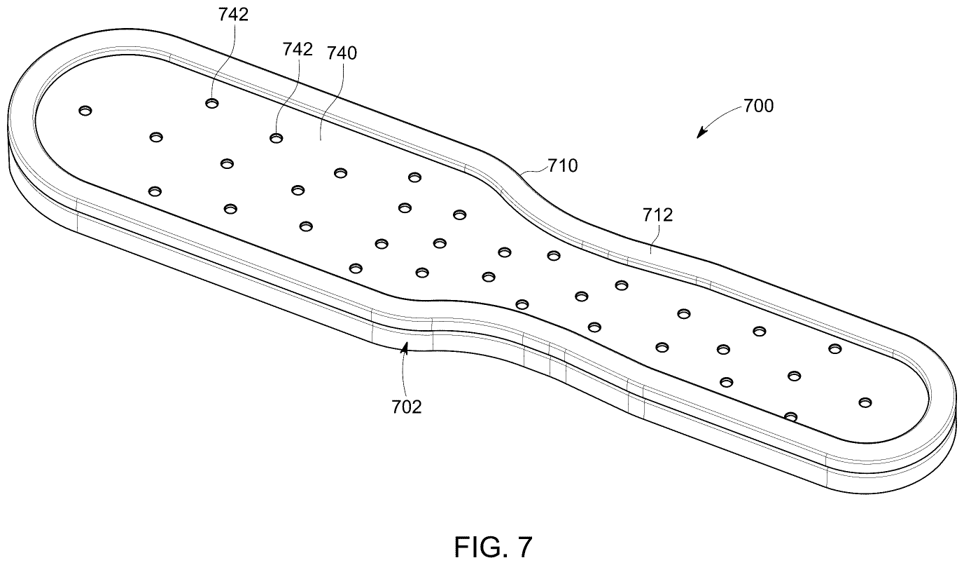

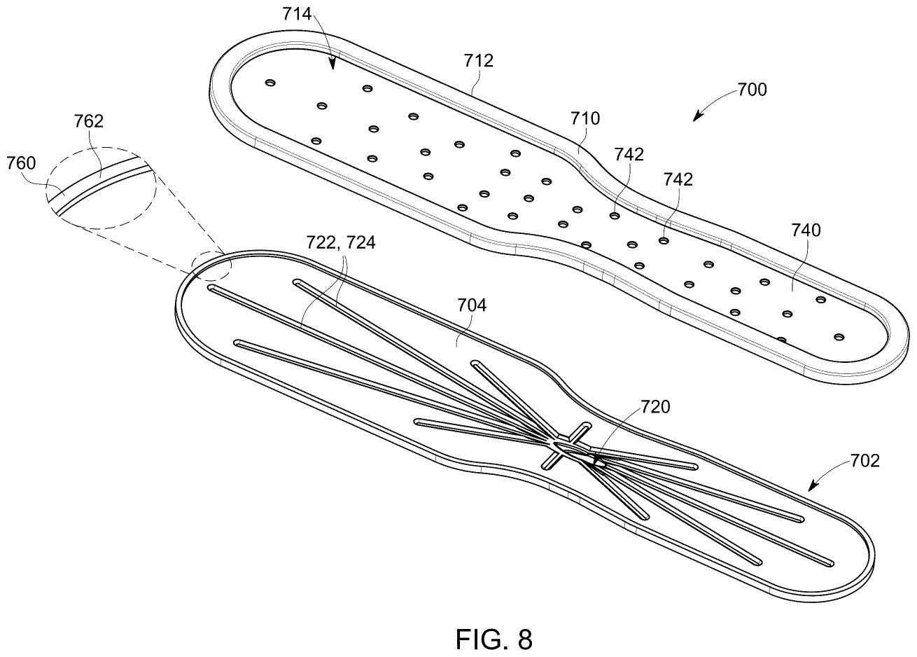

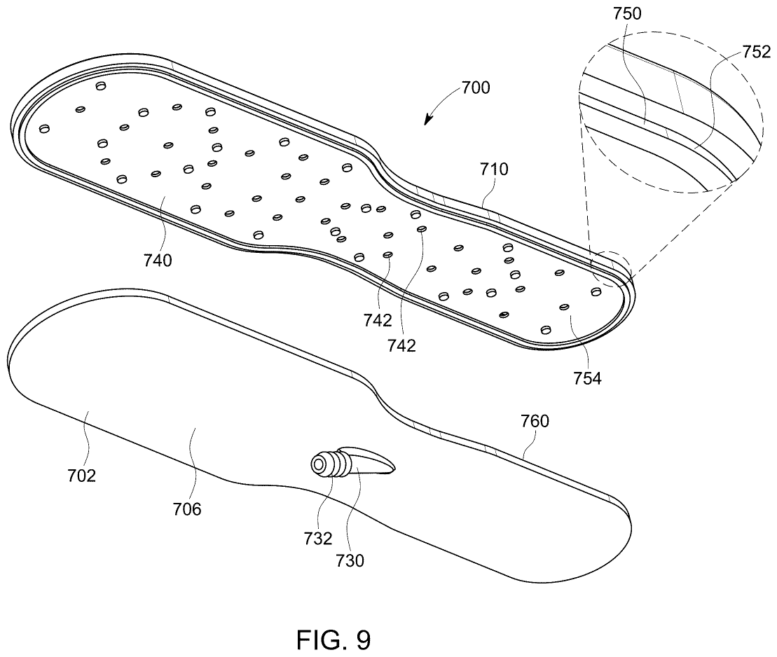

Referring to FIGS. 7 to 9, a pad 700 according to some alternative embodiments of the disclosure, is shown. The pad 700 is similar to the pad 100 and includes a panel body 702 similar to the panel body 102. As shown, the panel body 702 includes a first surface 704 and a second surface 706 arranged opposite to the first surface 704. To provide an exit of the urine from the pad 700, the panel body 702 defines an exit port 720 extending from the first surface 704 to the second surface 706. Moreover, the panel body 702 defines a plurality of elongated grooves 722 similar to the plurality of grooves 122. Each of the plurality of grooves 722 defines an elongated channel 724 with one end attached to the exit port 720 to allow a flow of urine to the exit port 720 from the first surface 704. Additionally, the panel body 702 includes a bead 740 extending along an outer edge of the panel body 702. Also, a valve 730 of the pad 700 is similar to the valve 130 and extends downwardly and outwardly of the second surface 706 of the panel body 702. The valve 730 is arranged connected with the exit port 730 to receive the urine from the exit port and direct the urine to the external bag 204 via the external tube 202. Similar to the valve 130, ribs 732 are arranged at an outer surface of the valve 730.

Moreover, the pad 700 includes a cover layer 740, i.e., outer layer, adapted to be removably attached to the panel body 702. Accordingly, the cover layer 740 may act as a disposable liner and can be changed. As shown, the cover layer 740 includes a plurality of holes 742 extending through a thickness of the cover layer 740 and is adapted to removably cover the panel body 702 from top. Accordingly, the urine flows to the panel body 702 from the cover layer 740 through the holes 742. Also, in the embodiment, the pad 700 includes ring structure 710 arranged attached and formed with the cover layer 740. The ring structure 710 is similar to the ring structure 110 except that the ring structure 710 is integrally formed with the cover layer 740. Accordingly, a chamber 714 to collect the urine is defined between an upper surface of the cover layer 740 and the ring structure 710. In some embodiments, an outer edge of the cover layer 740 is arranged underneath the ring structure 710 to form a mechanical seal and prevent leakage between the outer layer 740 and the panel body 702. For example, the ring structure 710 is separate from the cover layer 740, and in such a case, in the assembly of the panel body 702, the cover layer 740, and the ring structure 740, the outer edge of the cover layer 740 is arranged underneath the ring structure 710 and between the panel body 702 and the ring structure 710, and the ring structure 710 is engaged with the panel body 702.

To facilitate the removable attachment of the cover layer 740 with the panel body 702, the cover layer 740 or the ring structure 710 includes an engagement structure 750, for example a slot 752 define by a lower surface 754 of the cover layer 740 or the ring structure 710 and extending along an outer edge of the cover layer 740. Similarly, the panel body 702 includes a retention structure 760, for example, a bead 762, extending along an outer edge of the panel body 702, and upwardly from the first surface 704 of the panel body 702. In the engagement of the panel body 702 and the cover layer 740 or the ring structure 710, the engagement structure 750 is arranged connected with the retention structure 760 i.e., the bead 762 is arranged inside the slot 752 and snap fitted inside the slot 752. In embodiments, the outer/cover layer 740, the panel body 702, the ring structure 710, and the valve 730 may be made of materials similar to material described with reference to pad 100,

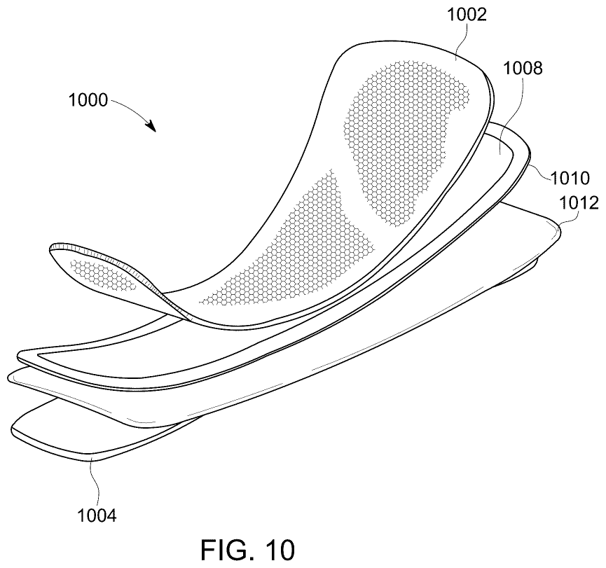

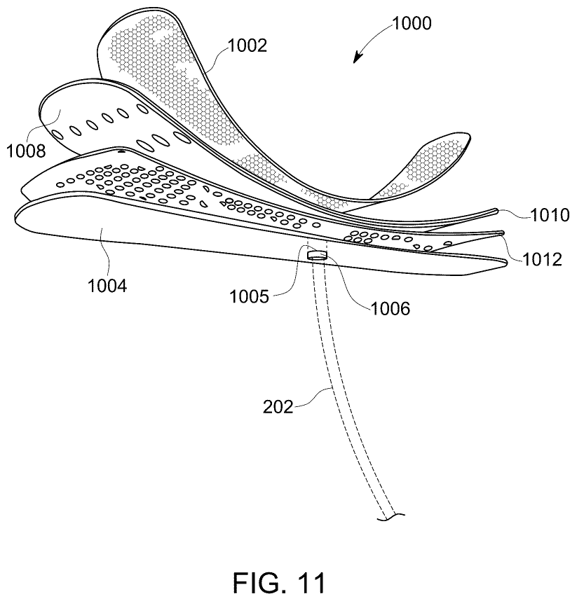

Referring to FIGS. 10 to 12, a pad 1000 according some alternative embodiment is disclosed. The pad 1000 includes an outer/cover layer 1002 i.e., first layer 1002 arranged to contact/face the skin of the user when the pad 1000 is worn by a wearer. In the embodiment, the first layer 1002 is made of synthetic layer and is a perforated layer, and allows passage of urine. Further, the pad 1002 includes a second layer 1004 also referred to as a panel body 1004 arranged coupled to the first layer 1002, and configured to receive the urine from the first layer 1002. In the embodiments, the second layer i.e., the panel body 1004 is formed of plastic and is made of waterproof material. Also, the panel body 1004 includes an exit port 1005 through which the urine exits the pad 1000. To facilitate the urine to flow to an external bag via an external tube 202, the pad 1000 includes a valve 1006 attached to the exit port 1005 of the panel body 1004 and extending outwardly of the panel body 1004. In the embodiment, the valve 1006 is removably attached to the panel body 1004. The valve 1006 allows a flow of the urine away from the pad 1000, while preventing any flow of the water or other fluid inside the pad 1000 through the exit port 1005. In the embodiments, the external tube 202 is connected to the valve 1006 to direct a flow of the urine from the pad 1000 to the external bag.

Further, in some embodiments, the pad 1000 includes a cushion layer 1008 with a ring structure 1010 extending along an outer edge of the cushion layer 1008 and raised upwardly from a surface of the cushion layer 1008. In the embodiments, the first layer 1002 is arranged attached on the cushion layer 1008 such that the ring structure 1010 extends outwardly of the first layer 1002. The ring structure 1010 is configured to contact the skin of the person acts as a sealing structure to prevent any leakage of urine collected on the first layer. In embodiments, the ring structure 1010 is formed of synthetic fiber, and is compressible to allow a sealing contact with the skin surface. In embodiments, the cushion layer 1008 is also a porous layer to allow a passage of the urine therethrough, while the ring structure 1010 is made of waterproof material. In some embodiments, the pad 1000 may include an intermediate layer 1012 sandwiched between the cushion layer 1008 and the panel body 1004. Accordingly, the urine flow through the outer layer 1002, then through the cushion layer 1008, and is absorbed by the absorbent layer 1012. Excess urine is collected on the panel body 1004 i.e., between the absorbent layer 1012 and panel body 1004, and exit the pad 1000 through the exit port 1005 and valve 1006. The outer layer 1002, the panel body 1004, the ring structure 1010, and the valve 1006 may be made of materials similar to material described with reference to pad 100.

Accordingly, the pad, according to embodiments of the present disclosure, provides a non-absorbent drainage device that receives the urine, discharged by the wearer, into a drainage chamber, simply referred to as chamber, with grooves, and exits via a one-way valve to an external bag. Thus, the pad 100, 700, 1000 keep the skin dry of urine, provides a well sleep or resting time. The pad 100, 700, 1000 can be worn at any time of the day as well. The external bag of adequate capacity can be attached with the pad to prevent frequent changing to bags. This pad is eco-friendly and hypoallergenic, thus preventing skin rashes.

In some embodiments, the pad and/or one or more components of the pad may be manufactured via injection molding, compression molding, or TPU film welding. The pad is lightweight, hypoallergenic, reusable, and eco-friendly.

It should be understood that the foregoing description is only illustrative of the aspects of the disclosed embodiments. Various alternatives and modifications can be devised by those skilled in the art without departing from the aspects of the disclosed embodiments. Accordingly, the aspects of the disclosed embodiments are intended to embrace all such alternatives, modifications, and variances that fall within the scope of the appended claims. Further, the mere fact that different features are recited in mutually different dependent or independent claims does not indicate that a combination of these features cannot be advantageously used, such as a combination remaining within the scope of the aspects of the disclosed embodiments.

What is claimed is:

1. A pad for collecting and directing urine of a wearer, the pad comprising:
a panel body having an exit port to allow an exit of urine;
a valve attached to panel body and arranged to receive urine from the panel body, wherein the valve enables an engagement of an external tube to direct the urine received from the panel body via the exit port to an external bag; and
a ring structure extending along a periphery of the panel body and adapted to contact a skin of the wear to provide a sealing between the pad and the skin to prevent any leakage of the urine from the pad.

2. The pad of claim 1, wherein the panel body defines a plurality of grooves defining a plurality of channels to direction the urine to the exit port.

3. The pad of claim 1, wherein the ring structure is integrally formed with the panel body and extends upwardly from the panel body defining a chamber to retain the urine.

4. The pad of claim 1 further comprising an outer layer arranged engaged to the panel body, wherein the outer layer is a perforated layer to allow a flow the urine received from the wearer to the panel body.

5. The pad of claim 4, wherein the outer layer is integrally formed with the panel body.

6. The pad of claim 4, wherein the outer layer is removably and replaceably attached to the panel body.

7. The pad of claim 6, wherein the panel body includes a retention structure and the outer layer includes an engagement structure configured to be removably engaged with the retention structure to removably engage the outer layer with the panel body.

8. The pad of claim 7, wherein the engagement structure is a slot and the retention structure is a bead adapted to extend inside the slot.

9. The pad of claim 4, wherein the ring structure is attached to the outer layer and extends along an outer edge of the outer layer.

10. The pad of claim 1, wherein the valve is removably attached to the panel body.
